(19) United States
(12) Patent Application Publication
(10) Pub. No.: US 2010/0332417 A1
(43) Pub. Date: Dec. 30, 2010

(54) METHODS FOR THE DETECTION AND QUANTITATION OF THE P95 COMPONENT OF HER2/NEU (ERBB2)

(75) Inventor: Sarah S. Bacus, Hinsdale, IL (US)

Correspondence Address: K&L Gates LLP, P.O. Box 1135, CHICAGO, IL, 60690, US

(73) Assignee: TARGETED MOLECULAR DIAGNOSTICS, LLC, Westmont, IL (US)

(21) Appl. No.: 12/792,027

(22) Filed: Jun. 2, 2010

Related U.S. Application Data
(60) Provisional application No. 61/183,125, filed on Jun. 2, 2009.

(51) Int. Cl. A61K 39/395; G01N 33/53; G01N 33/574; G01N 33/567; A61K 31/5377; A61K 31/517; A61P 35/00; G06Q 90/00

(52) U.S. Cl. 705/500; 436/501; 435/7.23; 435/7.21; 424/130.1; 424/141.1; 424/133.1; 424/142.1; 514/234.5; 514/266.1; 514/266.24

(57) ABSTRACT

The present disclosure relates generally to methods for the detection and quantitation of the p95 component of HER2/neu (ERbB2). Such methods may comprise assaying the biological sample for expression of HER2 ICD; assaying the biological sample for expression of a HER2 ECD; quantitating an expressed amount of the HER2 ICD and an expressed amount of the HER2 ECD in the biological sample; and determining the amount of p95 expressed in the biological sample based on a difference between the expressed amount of the HER2 ICD and the expressed amount of the HER2 ECD. The methods of the present disclosure may be used to predict whether a subject will be responsive to a receptor tyrosine kinase inhibitor and/or may be used to select subjects for inclusion/exclusion in a clinical trial.

### BACKGROUND

The human-epidermal-growth-factor receptor 2 (HER2, also known as neu, ErbB2, and p185^{HER2}) is a transmembrane glycoprotein with an intracellular tyrosine kinase domain and an extracellular domain very similar to those of the epidermal-growth-factor receptor (EGFR). HER family members are typically involved in stimulating signaling pathways that promote multiple processes that are potentially cancer-promoting (e.g. proliferation, angiogenesis, cell motility and invasion, decreased apoptosis and induction of drug resistance). As such, over-expression of HER family members are frequently associated with many cancers including, for example, breast, lung, colorectal, ovarian, renal cell, bladder, head and neck cancers, glioblastomas, and astrocytomas.

The ErbB2 receptor has been shown to be proteolytically cleaved into an extracellular domain (ECD) of approximately 110 kDa and an intracellular domain (ICD) of approximately 95 kDa. The ECD has been identified in the medium of tumor cells in culture as well as breast tumors. Notably, the levels of circulating ECD in the sera of breast cancer patients has been correlated to a poor prognosis (Brandt-Rauf (1995) Mutat Res 333(1-2): 203-8; Colomer et al. (2000) Clin Cancer Res 6(6): 2356-62; Kandl et al. (1994) Br J Cancer 70(4): 739-42; Yamauchi et al. (1997) J Clin Oncol 15(7): 2518-25). The finding that the ECD may be an important prognostic factor in human disease suggests that the ICD (p95) may play a role in tumor progression since it is retained within a cell. Several studies have shown that p95 has higher tyrosine kinase activity than the full length receptor (Bargmann et al. (1988) Embo J 7(7): 2043-52; Di Fiore et al. (1987) Science 237(4811): 178-82; Segatto et al. (1988) Mol Cell Biol 8(12): 5570-4). Additionally, a positive correlation has been shown between p95 levels and lymph node metastasis in ErbB2 overexpressing tumors.

The post-translational processing of ErbB2 into its cleaved p95 and p110 products has important implications for ErbB2 targeted therapy. Monoclonal antibodies (mAbs) directed to the ECD of ErbB2 would not be expected to be effective against the p95 truncated receptor since it lacks the ECD that is a target for this therapy. Given that the level of p95 present in a tumor may affect tumor response to a particular therapeutic, a quantitative evaluation of p95 levels may represent an important prognostic marker for patient response.

### SUMMARY

The present disclosure provides methods for the detection and quantitation of the p95 component of HER2/neu (ERbB2). Such methods may be used to determine the amount (e.g., percentage or ratio) of HER2 ICD (e.g., HER2 ICD protein; an intracellular domain of HER2/neu) detected in a biological sample (e.g., a formalin fixed paraffin embedded tissue sample) that is present as the p95 cleavage product (e.g., produced from proteolytically cleavage of p185) and/or the p95 truncated translation product (e.g., formed through the use of an internal start codon (L e., Methionine 687)).

The present disclosure also provides methods for determining an amount of p95 (polynucleotide such as RNA or DNA, or protein) in a biological sample, by assaying the biological sample for expression of an intracellular domain (ICD) of HER2/neu (e.g., HER2 ICD); assaying the biological sample for expression of an extracellular domain (ECD) of HER2/neu (e.g., HER2 ECD); quantitating an expressed amount of the ICD of HER2/neu and an expressed amount of the ECD of HER2/neu in the biological sample; and determining the amount of p95 expressed in the biological sample based on a difference between the expressed amount of the ICD of HER2/neu and the expressed amount of the ECD of HER2/neu.

The present disclosure also provides methods for predicting responsiveness of a subject with a disease or disorder to a receptor tyrosine kinase inhibitor by obtaining a biological sample from the subject; assaying the biological sample for expression of a ICD of HER2/neu; assaying the biological sample for expression of a ECD of HER2/neu; quantitating an expressed amount of the ICD of HER2/neu and the expressed amount of the ECD of HER2/neu in the biological sample; calculating the amount of p95 in the biological sample based on a difference between the expressed amount of the ICD of HER2/neu and the ECD of HER2/neu; comparing the amount of p95 in the biological sample to an amount of p95 in a control sample or to a threshold; and determining that the subject is responsive to a receptor tyrosine kinase inhibitor where the amount of p95 expressed in the biological sample is less than the amount of p95 expressed in the control sample or less than the threshold, or determining that the subject is not responsive to a receptor tyrosine kinase inhibitor where the amount of p95 expressed in the biological sample is greater than the amount of p95 expressed in the control sample or above the threshold.

The present disclosure also provides methods for treating a subject with a disease or disorder with a receptor tyrosine kinase inhibitor by obtaining a biological sample from the subject; assaying the biological sample for expression of a ICD of HER2/neu; assaying the biological sample for expression of a ECD of HER2/neu; quantitating an expressed amount of the ICD of HER2/neu and an expressed amount of the ECD of HER2/neu in the biological sample; calculating the amount of p95 expressed in the biological sample based on a difference between the expressed amount of the ICD of HER2/neu and the expressed amount of the ECD of HER2/neu; comparing the amount of p95 in the biological sample to an amount of p95 in a control sample or to a threshold; determining that the subject is responsive to a receptor tyrosine kinase inhibitor where the amount of p95 expressed in the biological sample is less than the amount of p95 expressed in the control sample or less than the threshold; and administering the receptor tyrosine kinase inhibitor to the subject.

In an embodiment of any of the above described methods, the biological sample is a tumor biopsy. In an embodiment of any of the above described methods, the biological sample is an aspirate.

In an embodiment of any of the above described methods, the ICD of HER2/neu and the ECD of HER2/neu are assayed using a detectably labeled antibody specific for the ICD of HER2/neu and a detectably labeled antibody specific for the ECD of HER2/neu.

In an embodiment of any of the above described methods, the antibody is a monoclonal antibody.

In an embodiment of any of the above described methods, the label is a chromagen or fluorophore.

In an embodiment of any of the above described methods, the step of assaying is performed by immunohistochemistry (IHC) or western blot.

In an embodiment of any of the above-described methods, the step of quantitating HER2 ICD and HER2 ECD expression is preformed by image analysis.

In an embodiment of any of the above described methods, the step of quantitating the expression of the ICD of HER2/neu and the expression of the ECD of HER2/neu is preformed in a defined cellular area.

In an embodiment of any of the above described methods, the defined cellular area is the nucleus. In an embodiment of any of the above described methods, the defined cellular area is the cell membrane. In an embodiment of any of the above described methods, the defined cellular area is the cytoplasm. In an embodiment of any of the above described methods, the defined cellular area is the nucleus, cytoplasm and/or cell membrane.

In an embodiment of any of the above described methods, the amount of the expression of the ICD of HER2/neu and the amount of expression of the ECD of HER2/neu is quantitated from an average optical density (OD) of expression of the ICD of HER2/neu and the ECD of HER2/neu per pixel in the defined cellular area.

In an embodiment of any of the above described methods, the amount of the expression of the ICD of HER2/neu and the amount of expression of the ECD of HER2/neu is quantitated from an average OD determined on a per cell basis in the defined cellular area. In an embodiment of any of the above described methods, the average OD on a per cell basis is obtained by dividing the average OD for the defined cellular area by a number of nuclei in the defined cellular area.

In an embodiment of any of the above described methods, the average optical density is determined by using image analysis.

In an embodiment of any of the above described methods, the amount of p95 in the biological sample is determined by subtracting the average OD obtained from the ICD of HER2/neu from the average OD obtained from the ECD of HER2/neu.

In an embodiment of any of the above described methods, the ICD (intracellular domain, C-terminal domain) comprises an amino acid sequence as set forth in SEQ ID NO: 3, which corresponds to amino acid residues from position 676 to position 1255 in HER2/neu as set forth in SEQ ID NO: 1. In an embodiment of any of the above described methods, the extracellular domain (extracellular domain, N-terminal domain) comprises an amino acid sequence as set forth in SEQ ID NO: 2, which corresponds to amino acid residues 1-675 in HER2/neu as set forth in SEQ ID NO: 1.

In an embodiment of any of the above described methods, the receptor tyrosine kinase inhibitor is an antibody. In an embodiment of any of the above described methods, the antibody is a monoclonal antibody. In an embodiment of any of the above described methods, the monoclonal antibody is cetuximab (Erbitux®), panitumumab, zalutumumab, nimotuzumab or matuzumab.

In an embodiment of any of the above described methods, the receptor tyrosine kinase inhibitor is a small molecule inhibitor. In an embodiment of any of the above described methods, the small molecule inhibitor is gefitinib, erlotinib or lapatinib.

In an embodiment of any of the above described methods, the disease or disorder is cancer. In an embodiment of any of the above described methods, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In an embodiment of any of the above described methods, the subject is a cancer patient.

In an embodiment of any of the above described methods, the control sample is obtained from the same patient from which the biological sample was obtained. In an embodiment of any of the above described methods, the control sample is obtained from a different patient from which the biological sample was obtained. In an embodiment of any of the above described methods, the control sample is non-cancerous cells or tissue.

In an embodiment of any of the above described methods, the threshold is set as a maximum amount of p95 in a biological sample in which a subject (or average or two or more subjects) is responsive to treatment with a receptor tyrosine kinase inhibitor.

In some embodiments, the threshold is that amount (e.g., level) of p95 protein above which a patient is non-responsive to treatment with a receptor tyrosine kinase inhibitor.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 shows a schematic of intracellular forms of ErbB2 (p95) arising through two mechanisms. The first mechanism is the activation of matrix metalloproteinases that act on the ECD portion of the full-length p185 form of ErbB2 resulting in extracellular shedding of the ECD (p110) and the retention of the intracellular p95 fragment. Alternatively, a shorter form of ErbB2 can be formed through the use of an internal start codon (Methionine 687) that results in a truncated form of ErbB2 (p95) that lacks the ECD.

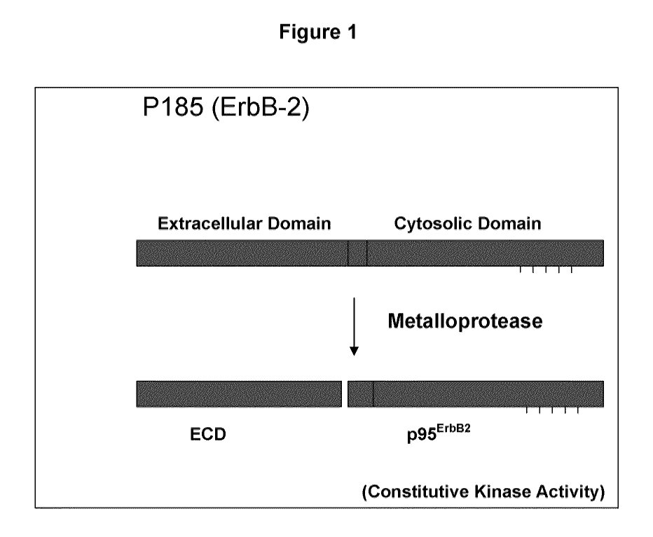

FIG. 2 shows an analysis of ErbB2 expression by IHC. Breast cancer patient samples were analyzed by IHC with antibodies specific to either the intracellular portion of ErbB2 (which detects both full-length and truncated ErbB2) or the extracellular portion of ErbB2 (which detects only full-length ErbB2). The patient that responded to Herceptin treatment had detectable ErbB2 using antibodies specific to both the ECD and the ICD of the protein. However, the non-responding patient had little detectable ECD of ErbB2 while still expressing copious ErbB2 intracellularly. This data indicates that the presence of the p95 portion of ErbB2 as indicated by little detection extracellularly and ample detection intracellularly, is a predictor for non-response to Herceptin treatment.

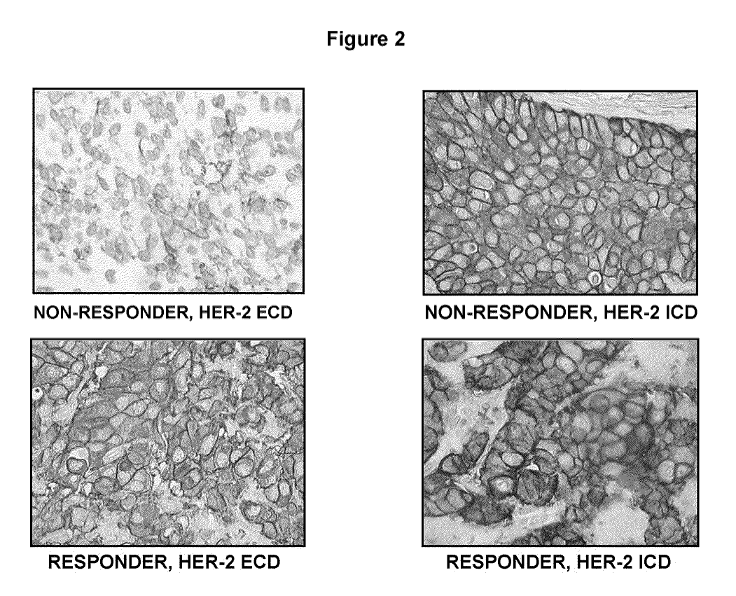

FIG. 3 shows expression of p95 ErbB2 in a breast cancer cell line (MDA-MB-453). Formalin-fixed, parrafin-embeded tumor cell line pellets were stained with either an antibody that is specific for the extracellular domain of ErbB2 (SP3, Ventana) or the intracellular domain of ErbB2 (Herceptest, Dako). AU-565 breast tumor cells show similar staining intensities with both antibodies indicating that the majority of ErbB2 is in the full-length form. MDA-MB-453 breast tumor cells show more intense staining with the antibody that is specific to the intracellular portion of ErbB2 (Herceptest) than the extracellular portion indicating that the majority of ErbB2 is in the p95 truncated form.

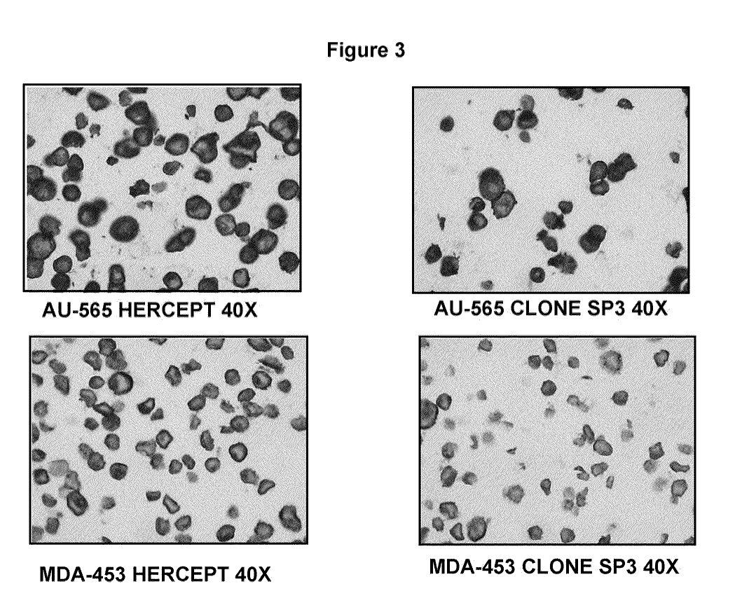

FIG. 4 shows a Western blot analysis for ErbB2 on lysates harvested from breast cancer patient samples using an antibody specific for the ECD of ErbB2. The main upper band is the full-length form of ErbB2 (p185) and the main lower band is the truncated form of ErbB2 (p95). In the table below, the Western blot results are data specific to analysis of tissue corresponding to the sample directly above in the Western blot. ICD and ECD are IHC scores for ErbB2 on these patient samples graded by an anatomical pathologist. TMD ICD and TMD ECD are densitometric OD values of the total ErbB2 and the extracellular portion of ErbB2, respectively, measured by image analysis of tissue. TMD p95 is calculated by subtracting the OD value of the p110 extracellular portion of ErbB2 (TMD ECD) from the OD value of the combined full-length and truncated forms of the intracellular portion of ErbB2 (TMD ICD).

FIG. 5 shows inter-day precision analysis for immunohistochemistry (IHC) performed on five cases in five consecutive experiments to demonstrate reproducibility and consistency of the assay. Bars represent the average of 5 experiments. Error bars represent standard deviation.

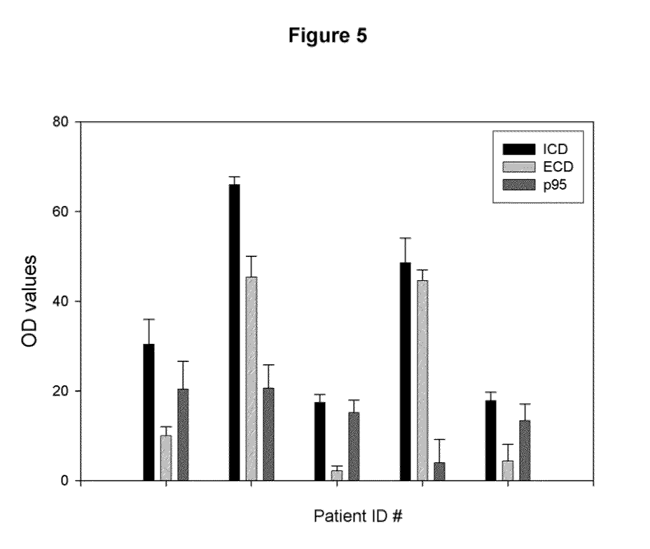

FIG. 6 shows analysis from an ErbB2 IHC and FISH. (A.) A 3+ tumor sample, showed low expression of p95 by imaging analysis. IHC with A0485 (OD_{ICD}=59) and TAB250-CBL772 (OD_{ECD}=51) showed equivalent staining (ODₚ₉₅=8). (B.) A 3+ tumor sample, showed high expression of p95 by imaging analysis. IHC with A0485 (OD_{ICD}=53) and TAB250-CBL772 (OD_{ECD}=8) showed a significant difference (ODₚ₉₅=45). Both samples had amplified ErbB2 by FISH analysis.

### DETAILED DESCRIPTION

The HER2/neu (ErbB2) gene encodes a protein of approximately 185 kDa involved in the EGFR signaling pathway. HER2/neu is often proteolytically cleaved in cancer resulting in the production of a p110 cleavage product (extracellular domain (ECD); N-terminal domain) and a p95 cleavage product (intracellular (ICD); C-terminal domain). Notably, loss of the ICD may be responsible for the ineffectiveness of antibody based therapy to HER2. Moreover, it has recently been shown that cancers that express the p95 component of HER2 exhibit an aggressive phenotype with an increased incidence of lymph node involvement and therapeutic resistance to tyrosine kinase inhibitors. Given that the p95 cleavage product is part of the full-length HER2 protein (p185), detection of p95 also detects the full-length intact (i.e., uncleaved) protein. As such, there has not existed a rapid and inexpensive method to detect p95 in a biological sample. Surprisingly, the inventors have discovered that the amount of p95 (e.g., amount of p95 protein) in a biological sample including, for example, a formalin fixed paraffin embedded tissue, may be determined by quantitating the difference in the expression of the ECD (or a fragment thereof) of HER2/neu and ICD (or a fragment thereof) of HER2/neu. Moreover, the inventors have demonstrated that conventional methods for accessing HER2 expression including, for example, immunohistochemistry (IHC) and fluorescence in situ hybridization (FISH) do not always correlate with p95 expression. Accordingly, these current methods may not provide an accurate assessment of a tumor sample such as its aggressiveness and responsiveness to treatment. Such methods of the disclosure may be used to determine (e.g., calculate) the amount of p95 in a biological sample. Additionally, the methods of the present disclosure may be used to predict whether a patient will be responsive to treatment with one or more receptor tyrosine kinase inhibitors and may be used to select subjects for inclusion/exclusion in a clinical trial.

The HER2 ICD may be proteolytically cleaved from the HER2 ECD resulting in the formation of p95 and/or the HER2 ICD may be produced as a p95 truncated protein through the use of an internal start codon. The methods of the disclosure may be used to determine the amount (e.g., level or percentage) of HER2 in a biological sample that is not associated with the HER2 ECD and/or determine the amount (e.g., percentage or ratio) of HER2 ICD detected in a biological sample that is present as the p95 cleavage product and/or the p95 truncated translation product. The amount (e.g., expression level) of p95 in a biological sample may be calculated by subtracting the OD value obtained for the detection of the extracellular portion (ECD) of HER2 from the OD value obtained for the detection of the combined full-length and truncated forms of the intracellular portion (ICD) of HER2. Notably, the analysis of p95 in HER2 over-expressing tumors may allow stratification of patients to receive either HER2-targeted antibody-based therapies such as Herceptin (e.g., targeted to the HER2 ECD) to patients that express little to no p95, or HER2-targeted small-molecular therapy such as Lapatinib (e.g. targeted to the HER2 ICD) to patients that express p95.

The present disclosure also provides methods for determining the amount (e.g., percentage or ratio) of HER2 ICD in a biological sample (e.g., cell and/or tissue) that is present in the proteolytically cleaved p95 form (p95 cleavage product) and/or the amount of p95 truncated translation product. Such methods may comprise assaying a biological sample for expression of HER2 ICD (e.g., with an antibody specific for the ICD of HER2/neu); assaying the biological sample for expression of HER2 ECD (e.g., with an antibody specific for the ECD of HER2/neu); quantitating an expressed amount of the HER2 ICD (e.g., average OD) and an expressed amount of the HER2 ECD (e.g., average OD) in the biological sample; determining the amount of p95 expressed in the biological sample based on a difference (e.g., subtraction) between the expressed amount of the HER2 ICD and the expressed amount of the HER2 ECD; and determining the amount of HER2 ICD that is in the proteolytically cleaved p95 form by comparing the amount of HER2 ICD detected in the biological sample to the amount of p95 detected in the biological sample. In some embodiments, the amount of HER2 ICD that is in the p95 cleaved form may be calculated by dividing the amount of HER2 ICD by the amount of p95 detected in the biological sample.

The present disclosure provides methods for determining an amount of p95 (e.g., amount of p95 protein) in a biological sample (e.g., a formalin fixed paraffin embedded tissue), by assaying the biological sample for expression of a ICD of HER2/neu with an antibody specific for the ICD of HER2/neu; assaying the biological sample for expression of a ECD of HER2/neu with an antibody specific for the ECD of HER2/neu; quantitating an expressed amount of the ICD of HER2/neu and an expressed amount of the ECD of HER2/neu in the biological sample; and determining the amount of p95 expressed in the biological sample based on a difference between the expressed amount of the ICD of HER2/neu and the expressed amount of the ECD of HER2/neu, wherein the amount of p95 in the biological sample is determined by subtracting the average OD obtained from the ICD of HER2/neu from the average OD obtained from the ECD of HER2/neu. In some embodiments, the biological sample may be from a tumor.

The present disclosure also provides methods for predicting responsiveness of a subject with a disease or disorder to a receptor tyrosine kinase inhibitor by obtaining a biological sample (e.g., a formalin fixed paraffin embedded tissue) from the subject; assaying the biological sample for expression of a ICD of HER2/neu with an antibody specific for the ICD of HER2/neu; assaying the biological sample for expression of a ECD of HER2/neu with an antibody specific for the ECD of HER2/neu; quantitating an expressed amount of ICD of HER2/neu and the expressed amount of the ECD of HER2/neu in the biological sample; calculating the amount of p95 in the biological sample based on a difference between the expressed amount of the ICD of HER2/neu and the ECD of HER2/neu; comparing the amount of p95 in the biological sample to an amount of p95 in a control sample or to a threshold and determining that the subject is responsive to a receptor tyrosine kinase inhibitor where the amount of p95 expressed in the biological sample is less than the amount of p95 expressed in the control sample or less than the threshold, or determining that the subject is not responsive to a receptor tyrosine kinase inhibitor where the amount of p95 expressed in the biological sample is greater than the amount of p95 expressed in the control sample or greater than the threshold, wherein the amount of p95 in the biological sample is determined by subtracting the average OD obtained from the ICD of HER2/neu from the average OD obtained from the ECD of HER2/neu. In some embodiments, the biological sample may be from a tumor.

The present disclosure also provides methods for treating a subject with a disease or disorder with a receptor tyrosine kinase inhibitor by obtaining a biological sample (e.g., a formalin fixed paraffin embedded tissue) from the subject; assaying the biological sample for expression of a ICD of HER2/neu with an antibody specific for the ICD of HER2/neu; assaying the biological sample for expression of a ECD of HER2/neu with an antibody specific for the ECD of HER2/neu; quantitating an expressed amount of the ICD of HER2/neu and an expressed amount of the ECD of HER2/neu in the biological sample; calculating the amount of p95 expressed in the biological sample based on a difference between the expressed amount of the ICD of HER2/neu and the expressed amount of the ECD of HER2/neu; comparing the amount of p95 in the biological sample to an amount of p95 in a control sample or to a threshold; determining that the subject is responsive to a receptor tyrosine kinase inhibitor where the amount of p95 expressed in the biological sample is less than the amount of p95 expressed in the control sample or less than the threshold; and administering the receptor tyrosine kinase inhibitor to the subject, wherein the amount of p95 in the biological sample is determined by subtracting the average OD obtained from the ICD of HER2/neu from the average OD obtained from the ECD of HER2/neu. In some embodiments, the biological sample may be from a tumor.

The present disclosure also provides methods for selecting subjects for inclusion/exclusion in a clinical trial by obtaining a biological sample (e.g., a formalin fixed paraffin embedded tissue) from the subject; assaying the biological sample for expression of HER2 ICD (e.g., with an antibody specific for the ICD of HER2/neu); assaying the biological sample for expression of HER2 ECD (e.g., with an antibody specific for the ECD of HER2/neu); quantitating the expressed amount of HER2 ICD and the expressed amount of HER2 ECD in the biological sample; determining the amount of p95 in the biological sample based on a difference between the expressed amount of the HER2 ICD and the HER2 ECD; selecting subjects for inclusion/exclusion in the clinical trial where the amount of p95 detected in their biological sample is less than (including equal to) a control level or threshold, or greater than a control level or threshold. In some embodiments, subject are excluded from the clinical trial where the amount of p95 detected in their biological samples is greater than a control level or threshold. In some embodiments, subjects are included in the clinical trial where the amount of p95 detected in their biological samples is equal to or less than a control amount or threshold. In some embodiments, the control amount of p95 is that amount of p95 detected in subjects that are tumor free.

The ECD of HER2/neu may comprise the amino acid sequence set forth in SEQ ID NO 2. The ICD may comprise the amino acid sequence as set forth in SEQ ID NO: 3. The ECD of HER2/neu and/or the ICD of HER2/neu may be a variant of the native protein including, for example, a biologically active variant, of the amino acid sequence as set forth in SEQ ID NO: 2 or 3, respectively. In some embodiments, the ICD of HER2/neu may be a protein having an approximate molecular weight of 95 kDa.

Variants of the ECD of HER2/neu and the ICD of HER2/neu may include biologically active variants which comprise an amino acid sequence that is at least 80%, more preferably 90%, still more preferably 95-99% similar to the native protein.

Guidance in determining which amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

Protein variants include glycosylated forms, aggregative conjugates with other molecules, and covalent conjugates with unrelated chemical moieties. Also, protein variants also include allelic variants, species variants, and muteins. Truncations or deletions of regions which do not affect the differential expression of the gene are also variants. Covalent variants can be prepared by linking functionalities to groups which are found in the amino acid chain or at the N- or C-terminal residue, as is known in the art.

It will be recognized in the art that some amino acid sequence of the ECD of HER2/neu and the ICD of HER2/neu can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Thus, the polypeptides of the present invention may include one or more amino acid substitutions, deletions or additions, either from natural mutations or human manipulation.

Amino acids in the polypeptides of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255: 306-312 (1992)).

Variants of the ECD of HER2/neu and the ICD of HER2/neu may include a protein possessing an amino acid sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to the ECD of HER2/neu and the ICD of HER2/neu (SEQ ID NO: 2 or 3, respectively). Preferably, this variant may possess at least one biological property in common with the native protein.

Sequence identity or percent identity is intended to mean the percentage of the same residues shared between two sequences, when the two sequences are aligned using the Clustal method [Higgins, et al., Cabios 8:189-191 (1992)] of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 [Dayhoff et al., in Atlas of Protein Sequence and Structure, Dayhoff, Ed., NDRF, Washington, Vol. 5, suppl. 3, p. 345, (1978)].

In an embodiment, the disease or disorder may be cancer. In an embodiment the cancer may be selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, glioma; parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

In an embodiment the cancer may be non-small cell lung cancer, pancreatic cancer, breast cancer, ovarian cancer, colorectal cancer, or head and neck cancer. In yet another embodiment the cancer may be a carcinoma, a tumor, a neoplasm, a lymphoma, a melanoma, a glioma, a sarcoma, or a blastoma.

In an embodiment the carcinoma may be selected from the group consisting of: carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiated carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In an embodiment the tumor may be selected from the group consisting of: astrocytic tumors, malignant mesothelial tumors, ovarian germ cell tumors, supratentorial primitive neuroectodermal tumors, Wilms tumors, pituitary tumors, extragonadal germ cell tumors, gastrinoma, germ cell tumors, gestational trophoblastic tumors, brain tumors, pineal and supratentorial primitive neuroectodermal tumors, pituitary tumors, somatostatin-secreting tumors, endodermal sinus tumors, carcinoids, central cerebral astrocytoma, glucagonoma, hepatic adenoma, insulinoma, medulloepithelioma, plasmacytoma, vipoma, and pheochromocytoma.

In an embodiment the neoplasm may be selected from the group consisting of: intraepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma.

In an embodiment the lymphoma may be selected from the group consisting of: nervous system lymphoma, AIDS-related lymphoma, cutaneous T-cell lymphoma, non-Hodgkin's lymphoma, lymphoma, and Waldenstrom's macroglobulinemia.

In an embodiment the melanoma may be selected from the group consisting of: acral lentiginous melanoma, superficial spreading melanoma, uveal melanoma, lentigo maligna melanomas, melanoma, intraocular melanoma, adenocarcinoma nodular melanoma, and hemangioma.

In an embodiment the sarcoma may be selected from the group consisting of: adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma.

In an embodiment the glioma may be selected from the group consisting of: glioma, brain stem glioma, and hypothalamic and visual pathway glioma.

In an embodiment the blastoma may be selected from the group consisting of: pulmonary blastoma, pleuropulmonary blastoma, retinoblastoma, neuroblastoma, medulloblastoma, glioblastoma, and hemangiblastomas.

### Detection and Quantitation of p95

A number of methodologies may be employed to detect and/or quantitate the amount (i.e., level) of p95 expression in a biological sample. Such expression of p95 may be detected at the protein level. Those skilled in the art will appreciate that the methods indicated below represent some of the preferred ways in which the level of p95 expression may be detected and/or quantitated and in no manner limit the scope of methodologies that may be employed. Those skilled in the art will also be able to determine operative and optimal assay conditions for each determination by employing routine experimentation. Such methods may include, but are not limited to, Western blots, ELISA, immunoprecipitation, immunofluorescence, flow cytometry and immunocytochemistry (IHC). In a preferred embodiment, p95 expression is detected and quantitated by IHC. Such methods of the present disclosure may comprise the detection and quantitation of the amount of the ICD of HER2/neu or fragment thereof and the amount of the ECD of HER2/neu or fragment thereof in a biological sample. In some embodiments, the ECD of HER2/neu may comprise amino acid residues 1-675 of SEQ ID NO: 1. In some embodiments, the ICD of HER2/neu may comprise amino acid resides 676-1255 of SEQ ID NO: 1.

Biological samples that may be used in any of the methods of the present disclosure may include tissues, cells, biological fluids and isolates thereof, isolated from a subject, as well as tissues, cells and fluids present within a subject (e.g., a patient). Preferably, biological samples comprise cells, most preferably tumor cells, that are isolated from body samples, such as, but not limited to, smears, sputum, biopsies, secretions, cerebrospinal fluid, bile, blood, lymph fluid, urine and faeces, or tissue which has been removed from organs, such as breast, lung, intestine, skin, cervix, prostate, and stomach. Biological samples may also include sections of tissues such as frozen sections taken for histological purposes.

In some embodiments, HER2 ICD and HER2 ECD expression may be quantitated by image analysis including, for example, computerized image analysis.

### Immunohistochemistry (IHC) Assays

The expression level of p95 in a biological sample may be determined by immunohistochemically staining cells in the sample using a detectably-labeled agent (e.g., an antibody) specific for the ECD of HER2/neu and a detectably-labeled agent (e.g., an antibody) specific for the ICD of HER2/neu. In a preferred embodiment, the agent is a monoclonal antibody and the detectable label is a chromagen or a fluorophore.

Both the ECD of HER2/neu and the ICD of HER2/neu can be separably detected using a specific agent, most preferably an antibody, that is itself detectably labeled, or using an unlabeled antibody specific for the ECD of HER2/neu and an unlabeled antibody specific for the ICD of HER2/neu and a second antibody that is detectably labeled and recognizes the unlabeled antibody specific for the ECD of HER2/neu or the unlabeled antibody specific for the ICD of HER2/neu. Alternatively, any molecule that can be detectably labeled and that specifically binds to ECD of HER2/neu or the ICD of HER2/neu can be used in the practice of the methods of the disclosure. In a preferred embodiment of the methods of the present disclosure, a two-component immunohistochemical staining system is used to differentially stain each of the ECD of HER2/neu and the ICD of HER2/neu and the tissue or cell sample so that the stained ECD of HER2/neu and the ICD of HER2/neu can be more readily distinguished from the counterstained tissue or cell sample.

In an exemplary method, the ECD of HER2/neu and the ICD of HER2/neu in the biological sample may be identified by adding a detectably-labeled primary antibody specific for the ECD of HER2/neu and a detectably-labeled primary antibody specific for the ICD of HER2/neu, or alternatively an unlabeled primary antibody and a detectably-labeled secondary antibody specific for the primary antibody. The antibodies may be incubated with the sample for a time to form complexes if the ECD of HER2/neu and/or the ICD of HER2/neu is present.

The complexes are then visualized by treating the sections with a stain including, for example, diaminobenizidine (DAB) stain under appropriate conditions. In a second step, the tissue may be counterstained with another optical enhancement factor, for example ethyl green. Although a staining technique using peroxidase and ethyl green is exemplary, other stains and optical enhancement factors are also suitable such as alkaline phosphatase based with specific chromagens such as Fast Red, Fast Green, etc. For example, the ECD of HER2/neu and the ICD of HER2/neu can be stained using diaminobenizidine (DAB) and the tissue or cell sample can be counterstained using ethyl green or methylene blue. Spectral studies have shown that the ethyl green stain offers good spectral separation from the DAB precipitate of the immunoperoxidase technique such that different features of the image can be readily separated by filtering it at two different wavelengths. This allows the image to be digitized into two separate images, one in which all the cell nuclei are optically enhanced (ethyl green or Fast Green) and one in which only those tissue areas with receptor staining (DAB) are optically enhanced. In a preferred embodiment, the images can be separated by a 600 nanometer (red) filter to produce an image of all of the counter stained area, and a 500 nanometer (green) filter to produce an image of only those tissue areas with the DAB precipitate staining.

In some embodiments, to further differentiate those stained cellular areas, an interactive threshold setting technique can be used where an operator visualizing the images can set a boundary on the areas under consideration. When the boundaries are set, the images may be formed by eliminating all parts of the image that are below the thresholds in optical density. A threshold may be set for the first image, and a second threshold may be set for the second image.

In some embodiments, where HER2 ICD and HER2 ECD are quantitated by NC, for example, using image analysis, the optical density (OD) threshold may be set at 0.5, 0.55, 0.60, 0.65, 0.70, 0.75, 0.80. 0.85, 0.90, 1.0 or greater.

In some embodiments, an OD threshold may be set such that those cellular areas (e.g., cell membrane, cytoplasm, or nucleus) that have staining below the threshold are eliminated from the final image on a per cell basis or on a per pixel basis.

Image processing may consist of first forming the mask image of the tissues under consideration with the red filter. This mask image may be stored and another image for expressed protein quantification may then be acquired by using the green filtered version of the same image. The effect of the filters in combination is to optically enhance (make darker) those areas of the tissue mask where tissue components are stained with DAB and to make lighter those tissue components with only green counterstain. An image analysis can then be performed using only those areas of the image that are stained and which are within the mask.

Red and green filters may be suitable for practice of the disclosure as well as DAB and green counterstain. This implementation shows a convenient and advantageous method for discriminating between two areas having counterstaining. It is recognized that there are various other staining or optical enhancement methods and filtering methods which can be used to optically enhance one particular area or feature over another cell feature such as Fast green, eosin, and the like.

Following immunohistochemical staining, the optical image of the tissue or cell sample generated by the computer-aided image analysis system may then be magnified under a light microscope and separated into a pair of images. Such equipment can include a light or fluorescence microscope, and image-transmitting camera and a view screen, most preferably also comprising a computer that can be used to direct the operation of the device and store and manipulate the information collected, most preferably in the form of optical density of certain regions of a stained tissue preparation. Image analysis devices useful in the practice of this disclosure include but are not limited to the CAS 200 (Becton Dickenson, Mountain View, Calif.), Chromavision or Tripath systems. The separated images are enhanced using a pair of optical filters, one having a maximum absorption corresponding to the stain and the other having a maximum absorption corresponding to the counterstain. In other embodiments of the method of the present disclosure, a plurality of image analysis filters are used to detect, differentiate, and quantitate the level of staining of different cellular proteins in various components (e.g., membrane, cytoplasm, and nucleus). In preferred embodiments, specific staining for the ECD of HER2/neu and the ICD of HER2/neu may be detected, measured and quantitated using image analysis equipment, defined herein as comprising a light or fluorescence microscope, and image-transmitting camera and a view screen, most preferably also comprising a computer that can be used to direct the operation of the device and also store and manipulate the information collected, most preferably in the form of optical density of certain regions of a stained tissue preparation. Image analysis devices useful in the practice of this disclosure include, but are not limited to, the CAS 200 system (Becton Dickenson, Mountain View, Calif.). From a digitized image, a nuclear or cytoplasmic image mask is formed by forming the image at one wavelength of light such as red wavelength or green optical filter. The tissue mask may be stored and a second filter is used to form another filtered image of the areas with the optical enhancement factor. Differentiation of cellular characteristics can be made by comparing the first image with the second image to obtain a quantification of material stained with the optical enhancement factor and thus, an assay of the amount of the particular target under study.

After immunohistochemical staining, a quantified measure of the percentage of expressing cells the ECD of HER2/neu and/or the ICD of HER2/neu can be taken by digitizing microscope images of stained samples, and converting light intensity values in each picture element (pixel) of the digitized image to optical density values, which correspond to the percentage of stained cell nuclei. More specifically, computerized image analysis can be used to determine from a digital grey scale image, a quantity of cells having a particular stain. The grey scale images are repfesentative of the amount of an optical enhancement factor, such as a chromagen, which binds to a specific target under study and thereby allows optical amplification and visualization of the target.

The present disclosure also includes methods for fixing cells and tissue samples for analysis. Generally, neutral buffered formalin may be used. Any concentration of neutral buffered formalin that can fix tissue or cell samples without disrupting the epitope can be used. Preferably, the method includes suitable amounts of phosphatase inhibitors to inhibit the action of phosphatases and preserve phosphorylation. Any suitable concentration of phosphatase inhibitor can be used so long as the biopsy sample is stable and phosphatases are inhibited, for example 1 mM NaF and/or Na3VO4 can be used. In one method a tissue sample or tumor biopsy may be removed from a patient and immediately immersed in a fixative solution which can and preferably does contain one or more phosphatase inhibitors, such as NaF and/or Na₃VO₄. Preferably, when sodium orthovanadate is used it is used in an activated or depolymerized form to optimize its activity. Depolymerization can be accomplished by raising the pH of its solution to about 10 and boiling for about 10 minutes. The phosphatase inhibitors can be dissolved in the fixative just prior to use in order to preserve their activity. Fixed samples can then be stored for several days or processed immediately. To process the samples into paraffin after fixing, the fixative can be thoroughly rinsed away from the cells by flushing the tissue with water. The sample can be processed to paraffin according to normal histology protocols which can include the use of reagent grade ethanol. Samples can be stored in 70% ethanol until processed into paraffin blocks. Once samples are processed into paraffin blocks, they can be analyzed histochemically for virtually any antigen that is stable to the fixing process.

In practicing the method of the present disclosure, staining procedures can be carried out by a technician in the laboratory. Alternatively, the staining procedures can be carried out using automated systems. In either case, staining procedures for use according to the methods of this disclosure are performed according to standard techniques and protocols well-established in the art.

The amount of p95 can then be quantitated by image analysis using the expressed amount of the ECD of HER2/neu and the ICD of HER2/neu in the biological sample. For example, the amount of the expression of the ICD of HER2/neu and the amount of expression of the ECD of HER2/neu may be quantitated from an average optical density (OD) of expression of the ICD of HER2/neu and the ECD of HER2/neu per pixel in a defined cellular area. Alternatively, the amount of the expression of the ICD of HER2/neu and the amount of expression of the ECD of HER2/neu may be quantitated from an average OD determined on a per cell basis in the defined cellular area. The average OD on a per cell basis may be obtained by dividing the average OD for the defined cellular area by a number of nuclei in the defined cellular area. The amount of p95 in the biological sample may then be determined by subtracting the average OD obtained from the ICD of HER2/neu from the average OD obtained from the ECD of HER2/neu.

### Protein Based Assays

The expression level of p95 in a biological sample may be determined by immunohistochemically staining cells in the sample using a detectably-labeled agent (e.g., an antibody) specific for the ECD of HER2/neu and a detectably-labeled agent (e.g., an antibody) specific for the ICD of HER2/neu. P95 expression may be quantified at the protein level using methods known in the art, for example using quantitative enzyme linked immunosorbent assays (“ELISA”). Methods for designing and using quantitative ELISA assays are well known in the art. These methods require use of monoclonal or polyclonal antibodies that are specific for each of the ECD of HER2/neu and the ICD of HER2/neu.

Suitable monoclonal antibodies may be prepared by standard hybridoma methods, using differential binding assays to ensure that the antibodies are specific for each of the ECD of HER2/neu and the ICD of HER2/neu and do not show cross-reactivity between related proteins. Alternatively, suitable monoclonal antibodies may be prepared using antibody engineering methods such as phage display. Methods for obtaining highly specific antibodies from antibody phage display libraries are known in the art, and several phage antibody libraries are commercially available from, for example, MorphoSys (Martinsried, Germany), Cambridge Antibody Technology (Cambridge UK) and Dyax (Cambridge Mass.). Suitable phage display methods are described, for example, in U.S. Pat. Nos. 6,300,064, 5,969,108 and “Antibody Engineering,” McCafferty et al. (Eds.)(IRL Press 1996). Once the antibody heavy and light chain genes are recovered from the phage antibodies, antibodies in any suitable format may be prepared, e.g. whole antibodies, Fab, scFv, etc.

Other antibody preparations may also be used, for example Camelid antibodies, which contain only heavy immunoglobulin chains (e.g., Muyldermans, et al. J. Biotechnol. June; 74(4):277-302 (2001)). Other antibody formats are described, for example in “Antibody Engineering,” McCafferty et al. (Eds.) (IRL Press 1996).

Polyclonal antibodies specific for each of the ECD of HER2/neu and the ICD of HER2/neu may also be prepared using traditional animal-based methods. Peptides derived from each of the ECD of HER2/neu and the ICD of HER2/neu can be conjugated at their N- or C-termini to carrier proteins such as bovine serum albumin (BSA) or keyhole limpet hemocyanin (KLH) and used to immunize animals, such as rabbits, using well-known immunization regimes. Specific polyclonal antibodies can be obtained from the serum of the animal by, for example, affinity chromatography over a matrix containing the peptide used for immunization bound to a solid support.

An ELISA assay may be used to quantitate the level of expression of each of the ECD of HER2/neu and the ICD of HER2/neu. Many ELISA applications and formats have been described. Various sources provide discussion of ELISA chemistry, applications, and detailed protocols (See e.g., Crowther, “Enzyme-Linked Immunosorbent Assay (ELISA),” in Molecular Biomethods Handbook, Rapley et al., pp. 595-617, Humana Press, Inc., Totowa, N.J. (1998); Harlow and Lane (eds.), Antibodies. A Laboratory Manual, Cold Spring Harbor Laboratory Press (1988); Ausubel et al. (eds.), Current Protocols in Molecular Biology, Ch. 11, John Wiley & Sons, Inc., New York (1994); and Laurino et al., Ann. Clin. Lab Sci., 29(3):158-166 (1999)).

In one embodiment of the present disclosure, an ELISA based method is provided in which an antibody specific for the ECD of HER2/neu and an antibody specific for the ICD of HER2/neu is first immobilized on a solid support (e.g. in a microtiter plate well). Protein samples are then added to the plate from either a control or biological sample containing the ECD of HER2/neu and the ICD of HER2/neu. Each of the ECD of HER2/neu and the ICD of HER2/neu is then bound to antibody and can be detected and quantitated by the use of an antibody-enzyme conjugate capable of binding to another region of each of the ECD of HER2/neu and the ICD of HER2/neu (not bound by the first antibody) and producing a quantifiable signal. In some embodiments, the amount of antigen present is directly proportional to the amount of enzyme reaction product produced after the addition of an appropriate enzyme substrate.

As indicated previously, enzymes commonly used in ELISAs include horseradish peroxidase (HRPO), urease, alkaline phosphatase, glucoamylase and β-galactosidase. Protocols for the preparation of suitable antibody-enzyme conjugates are well known in the art. The present disclosure provides methods for the preparation of an antibody-enzyme (i.e., HRPO enzyme) conjugate that specifically recognizes the antigens of interest (i.e., CD4 and CD40) for use in an immunoassay (e.g., ELISA). The method provided herein, as those of skill in the art will recognize other methods for antibody-enzyme conjugation that find use with the present disclosure.

Conjugation of enzymes to antibodies involves the formation of a stable, covalent linkage between an enzyme (e.g., HRPO or alkaline phosphatase) and the antibody (e.g., the anti-CD4 and anti-CD40 antibodies), where neither the antigen-binding site of the antibody nor the active site of the enzyme may be functionally altered.

The conjugation of antibody and HRPO is dependent on the generation of aldehyde groups by periodate oxidation of the carbohydrate moieties on HRPO (Nakane and Kawaoi, J. Histochem. Cytochem., 22:1084-1091 (1988)). Combination of these active aldehydes with amino groups on the antibody forms Schiff bases that, upon reduction by sodium borohydride, become stable.

Protocols to make antibody-enzyme conjugates using urease or alkaline phosphatase enzymes are also known in the art (Healey et al., Clin. Chim. Acta 134:51-58 (1983); Voller et al., Bull. W.H.O., 53:55-65 (1976); and Jeanson et al., J. Immunol. Methods 111:261-270 (1988)). For urease conjugation, cross-linking of the urease enzyme (e.g., Urease Type VII, Sigma No. U0376) and antibody using m-maleimidobenzoyl N-hydroxysuccinimide ester (MBS) may be achieved through benzoylation of free amino groups on the antibody. This may be followed by thiolation of the maleimide moiety of MBS by the cysteine sulthydryl groups of urease. To prepare an alkaline phosphatase-antibody conjugate, a one-step glutaraldehyde method may be used (e.g., Voller et al., Bull. W.H.O., 53:55-65 (1976)). This antibody-alkaline phosphatase conjugation protocol uses an enzyme immunoassay grade of the alkaline phosphatase enzyme.

The end product of an ELISA may be a signal typically oberved as the development of color or fluorescence. Typically, this signal may be read (i.e., quantitated) using a suitable spectrocolorimeter (i.e., a spectrophotometer) or spectrofluorometer. The amount of color or fluorescence is directly proportional to the amount of immobilized antigen. In some embodiments of the present disclosure, the amount of antigen in a sample (e.g., the amount of the ECD of HER2/neu and the amount of the ICD of HER2/neu) may be quantitated by comparing results obtained for the sample with a series of control wells containing known concentrations of the antigen (i.e., a standard concentration curve). A negative control may also be included in the assay system.

It is contemplated that any suitable chromogenic or fluorogenic substrates will find use with the enzyme-conjugated antibodies of the present disclosure. In some embodiments of the present disclosure, the substrate p-nitrophenyl phosphate (NPP) in diethanolamine may be the preferred substrate for use in colorimetric ELISA methods, and 4-methylumbelliferyl phosphate (MUP) may be the preferred alkaline phosphatase substrate in fluorometric ELISA methods. Conjugated antibodies can include radioisotopes, fluorophores, enzymes, luminescers, or visible particles (e.g., colloidal gold and dye particles). These and other labels are well known in the art (e.g., U.S. Pat. Nos. 3,766,162; 3,791,932; 3,817,837; 3,996,345; and 4,233,402).

The present disclosure also provides various ELISA protocols for the detection and/or quantitation of each of the ECD of HER2/neu and the ICD of HER2/neu. In one embodiment, the present disclosure provides a “direct ELISA” for the detection of each of the ECD of HER2/neu and the ICD of HER2/neu. In some embodiments, the antigen of interest in a sample (i.e., the ECD of HER2/neu and the ICD of HER2/neu) may be bound (along with unrelated antigens) to the solid support (e.g., a microtiter plate well). The immobilized antigen is then directly detected by the antigen-specific enzyme-conjugated antibody, also provided by the present disclosure. Addition of an appropriate detection substrate results in color development or fluorescence that is proportional to the amount of each of the ECD of HER2/neu and the ICD of HER2/neu present in the well.

In another embodiment, the present disclosure provides an indirect ELISA for the detection of each of the ECD of HER2/neu and the ICD of HER2/neu in a sample. In this embodiment, antigen of interest in a sample may be immobilized (along with unrelated antigens) to a solid support (e.g., a microtiter plate well) as in the direct ELISA, but may be detected indirectly by first adding an antigen-specific antibody, then followed by the addition of a detection antibody specific for the antibody that specifically binds the antigen, also known as “species-specific” antibodies (e.g., a goat anti-rabbit antibody), which are available from various manufacturers known to one in the art (e.g., Santa Cruz Biotechnology; Zymed; and Pharmingen/Transduction Laboratories).

In another embodiment, the present disclosure provides “sandwich ELISA” methods, in which the antigen in a sample may be immobilized on a solid support by a “capture antibody” that has been previously bound to the solid support. In general, the sandwich ELISA method may be more sensitive than other configurations, and is capable of detecting 0.1-1.0 ng/ml protein antigen. As indicated above, the sandwich ELISA method involves pre-binding the “capture antibody” which recognizes the antigen of interest (i.e., the ECD of HER2/neu and the ICD of HER2/neu) to the solid support (e.g., wells of the microtiter plate). In some embodiments, a biotinylated capture antibody may be used in conjunction with avidin-coated wells. Test samples and controls are then added to the wells containing the capture antibody. If antigen is present in the samples and/or controls, it is bound by the capture antibody.

In some embodiments, after a washing step, detection of antigen that has been immobilized by the capture antibody may be detected directly (i.e., a direct sandwich ELISA). In other embodiments, detection of each of the ECD of HER2/neu and the ICD of HER2/neu that has been immobilized by the capture antibody may be detected indirectly (i.e., an indirect sandwich ELISA). In the direct sandwich ELISA, each of the ECD of HER2/neu and the ICD of HER2/neu is detected using a specific enzyme-conjugated antibody. In the indirect sandwich ELISA, each of the ECD of HER2/neu and the ICD of HER2/neu is detected by using an antibody specific for each of the ECD of HER2/neu and the ICD of HER2/neu, which is then detected by another enzyme-conjugated antibody which binds the antigen-specific antibody, thus forming an antibody-antigen-antibody-antibody complex. In both the direct and indirect sandwich ELISAs, addition of a suitable detection substrate results in color development or fluorescence that is proportional to the amount of each of the ECD of HER2/neu and the ICD of HER2/neu that is present in the well.

In the sandwich ELISA, the capture antibody used is typically different from the second antibody (the “detection antibody”). The choice of the capture antibody is empirical, as some pairwise combinations of capture antibody and detection antibody are more or less effective than other combinations. The same monoclonal antibody must not be used as both the capture antibody and the conjugated detection antibody, since recognition of a single epitope by the capture antibody will preclude the enzyme-conjugated detection antibody from binding to the antigen. However, in some embodiments, two different monoclonal antibodies that recognize different epitopes are used in this assay. In other embodiments, the same polyclonal antibody preparation is used as both the capture antibody and conjugated detection antibody, since multiple epitopes are recognized in the pool of polyclonal antibody species.

Furthermore, it is not intended that the present disclosure be limited to the direct ELISA and sandwich ELISA protocols particularly described herein, as the art knows well numerous alternative ELISA protocols that also find use in the present disclosure (see, e.g., Crowther, “Enzyme-Linked Immunosorbent Assay (ELISA),” in Molecular Biomethods Handbook, Rapley et al., pp. 595-617, Humana Press, Inc., Totowa, N.J. (1998); and Ausubel et al. (eds.), Current Protocols in Molecular Biology, Ch. 11, John Wiley & Sons, Inc., New York (1994)). Thus, any suitable ELISA method including, but not limited to, competitive ELISAs also find use with the present disclosure.

In yet a further embodiment of the present disclosure, an ELISA amplification system is provided. These amplification systems produce at least 10-fold, and more preferably, a 500-fold increase in sensitivity over traditional alkaline phosphatase-based ELISAs. In one preferred embodiment of the ELISA amplification protocol, bound alkaline phosphatase acts on an NADPH substrate, whose reaction product initiates a secondary enzymatic reaction resulting in a colored product. Each reaction product from the first reaction initiates many cycles of the second reaction in order to amplify the signal (see e.g., Bio-Rad ELISA Amplification System, Cat. No. 19589-019).

In yet another embodiment of the present disclosure, expression of each of the ECD of HER2/neu and the ICD of HER2/neu may be measured and quantitated by Western Blot analysis. Briefly, protein samples may be electrophoresed on an acrylamide gel and transferred to a membrane such as nitrocellulose or PVDF. The blot is detected with an antibody specific for each of the ECD of HER2/neu and the ICD of HER2/neu. These primary antibodies are then detected, for example, with labeled secondary antibodies. The fluorescence intensity of the dye is measured for both a test and control sample and the ratio of the intensity indicates the ratio of the two proteins.

The amount of p95 can be quantitated from the expressed amount of the ECD of HER2/neu and the ICD of HER2/neu in the biological sample. For example, the amount of the expression of p95 in the biological sample may be determined by subtracting the amount of the ICD of HER2/neu from the amount of the ECD of HER2/neu.

### Methods for Predicting Responsiveness to a Receptor Tyrosine Kinase Inhibitor

The present disclosure includes methods for predicting responsiveness of a subject with a disease or disorder to a receptor tyrosine kinase inhibitor by obtaining a biological sample from the subject; assaying the biological sample for expression of a ICD of HER2/neu; assaying the biological sample for expression of a ECD of HER2/neu; quantitating an expressed amount of the ICD of HER2/neu and the expressed amount of the ECD of HER2/neu in the biological sample; calculating the amount of p95 in the biological sample based on a difference between the expressed amount of the ICD of HER2/neu and the ECD of HER2/neu; and determining that the subject is responsive to a receptor tyrosine kinase inhibitor where the amount of p95 expressed in the biological sample is less than the amount of p95 expressed in a control sample or determining that the subject is not responsive to a receptor tyrosine kinase inhibitor where the amount of p95 expressed in the biological sample is greater than the amount of p95 expressed in a control sample or determining that the subject is not responisive to a receptor tyrosine kinase inhibitor where p95 is detected in the biological sample. The subject may be predicted to be responsive to the receptor tyrosine kinase inhibitor where the amount of p95 detected and quantitated in the biological sample is greater than the amount of p95 detected in a control biological sample obtained from the same subject from which the biological sample was obtained or a different subject from which the biological sample was obtained.

In some embodiments, the subject may be predicted to not be responsive to the receptor tyrosine kinase inhibitor when p95 is detected in the biological sample. According to Scaltriti et al. (2007) JNCI 99(8): 628-638, immune-detection of p95 in tumor samples compared to immunoblotting technique (Western blot) held a positive predictive value of 100% and a negative predicted value of 94% when compared to the previously reported prevalence of p95HER2 in the HER2-overexpressing patient population.

The analysis of p95 in HER2 over-expressing tumors may allow stratification of patients to receive either HER2-targeted antibody-based therapies (e.g., targeted to the HER2 ECD) to patients that express little to no p95, or HER2-targeted small-molecular therapy such as Lapatinib (e.g. targeted to the HER2 ICD) to patients that express p95.

In some embodiments, the subject may be predicted to be responsive to the receptor tyrosine kinase inhibitor where the amount of p95 (e.g., p95 protein) in the biological sample is substantially equal to or less than the amount of HER2 ICD detected in the biological sample. Alternatively, the subject may be predicted to be non-responsive to the receptor tyrosine kinase inhibitor where the amount of p95 in the biological sample is substantially equal to or more than the amount of HER2 ICD detected in the biological sample.

In some embodiments, the subject may be predicted to be non-responsive to the receptor tyrosine kinase inhibitor where the amount of p95 (e.g., amount of p95 protein) in the biological sample is 5%, 10%, 15%, 20%, 25%, 30%, 35%, 40%, 45%, 50%, 55%, 60%, 65%, 70%, 75%, 80%, 85%, 90%, 95%, 100% or more than the amount of HER2 ICD detected in the sample.

In some embodiments, the subject may be predicted to be responsive to the receptor tyrosine kinase inhibitor where the amount of p95 in the biological sample is equal to or less than a threshold or an amount from a control sample. In other embodiments, the subject may be predicted to be non-responsive to the receptor tyrosine kinase inhibitor where the amount of p95 in the biological sample is equal to or more than a threshold or an amount from a control sample.

In some embodiments, the control sample may be obtained from the same subject from which the biological sample was obtained. In other embodiments, the control sample may be obtained from a different subject from which the biological sample was obtained. Alternatively, the subject may be predicted to be responsive to the receptor tyrosine kinase inhibitor where the amount of p95 in the biological sample is less than a threshold. Such threshold may be set as an amount of p95 present in a sample above which a subject does not respond to treatment with a receptor tyrosine kinase inhibitor (i.e., the threshold is set as a maximum amount of p95 in a biological sample in which a subject is responsive to treatment with a receptor tyrosine kinase inhibitor). A threshold amount of p95 above which a subject is not predicted to respond to treatment with a receptor tyrosine kinase inhibitor may be determined by calculating an amount (e.g., an average amount or an approximate amount) of expression of p95 in tumor samples obtained from one or more subjects that respond to treatment with a receptor tyrosine kinase inhibitor and an amount (e.g., an average amount or an approximate amount) of expression of p95 in tumor samples obtained from one or more subjects that do not respond to treatment with a receptor tyrosine kinase inhibitor. Such a threshold above which a subject is predicted to not respond to treatment with a receptor tyrosine kinase inhibitor may be determined by IHC in a field of a defined size.

In some embodiments, the receptor tyrosine kinase inhibitor may be an antibody including, for example, a monoclonal antibody. Monoclonal antibodies may include, but are not limited to, cetuximab (Erbitux®), panitumumab, zalutumumab, nimotuzumab or matuzumab. In other embodiments, the receptor tyrosine kinase inhibitor is a small molecule inhibitor. Small molecule inhibitors may include, but are not limited to, gefitinib, erlotinib or lapatinib.

A determination of whether a subject will be predicted to be responsive to a receptor tyrosine kinase inhibitor may be used to direct a therapeutic regimen for a particular disease or disorder including, for example, cancer. Such methods may comprise obtaining a biological sample from the subject; assaying the biological sample for expression of a ICD of HER2/neu; assaying the biological sample for expression of a ECD of HER2/neu; quantitating an expressed amount of the ICD of HER2/neu and an expressed amount of the ECD of HER2/neu in the biological sample; calculating the amount of p95 expressed in the biological sample based on a difference between the expressed amount of the ICD of HER2/neu and the expressed amount of the ECD of HER2/neu; determining that the subject is responsive to a receptor tyrosine kinase inhibitor where the amount of p95 expressed in the biological sample is less than the amount of p95 expressed in a control sample; and administering the receptor tyrosine kinase inhibitor to the subject.

This disclosure is further illustrated by the following examples which are provided to facilitate the practice of the disclosed methods. These examples are not intended to limit the scope of the disclosure in any way.

### Examples

### Example 1

### Detection and Quantitation of p95 by Immunohistochemistry

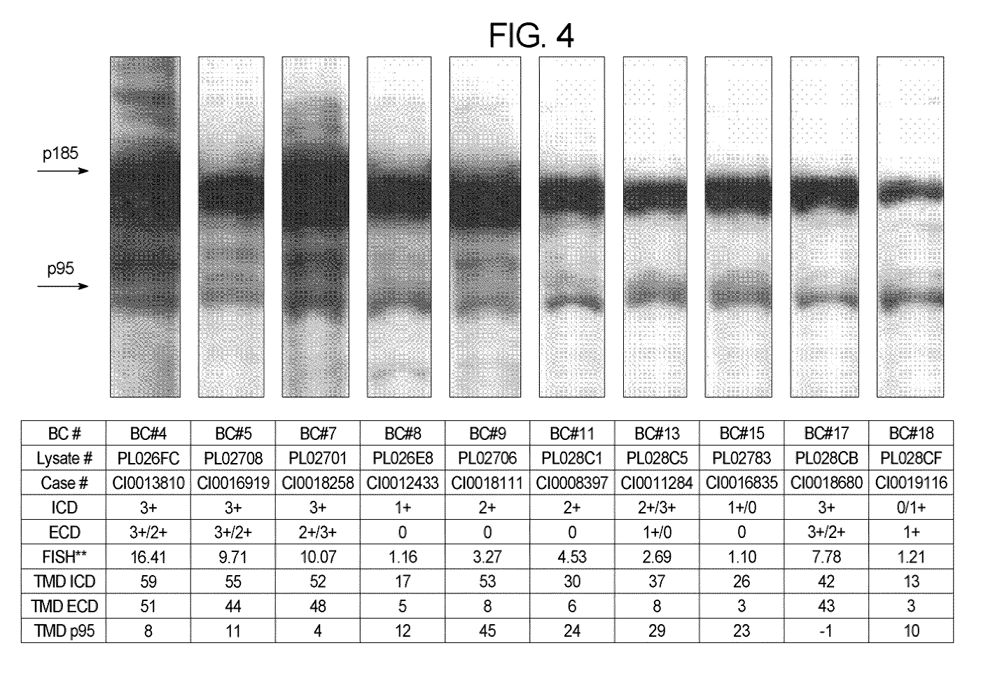

P95 may be detected in a biological sample (e.g., a cancer) by immunohistochemistry (IHC). Alternatively, p95 may be detected by Western Blot (see, e.g., FIG. 4). In an exemplary method, p95 may be detected and quantitated in a paraffin embedded tissue sample. Such methods may comprise deparaffinizing the tissue section, retrieval of an antigen (i.e., p95), staining the antigen, counterstaining the tissue section and image analysis.

A tissue section may be deparaffinized by any known method in the art. In an exemplary method, slides were dried in a 60° C. oven for 1.5 hours. Next, dried slides were placed in a plastic slide holder which was subsequently placed into the first change of xylene for 10 minutes. The slide holder was then placed into the second change of xylene for 10 minutes. Next, the slide holder was placed into the first change of absolute alcohol for 10 minutes followed by a second change of absolute alcohol for 10 minutes. The slide holder was then placed into 95% alcohol for 5 minutes. Next, the slide holder was placed into 70% alcohol for 5 minutes. The slides were then placed into an empty plastic staining dish and rinsed in running deionized water for 5 minutes.

After the tissue sections are deparaffinized, one or more epitopes of p95 may be retrieved for detection (e.g., using a Biocare Decloaking Chamber). Slides were antigen retrieved with Dako Target Retrieval Solution in a DeCloaking Chamber (Biocare Medical) with an SP1 time of 30 seconds and SP2 time of 10 seconds. Next, slides were washed immediately in running deionized water for 3 minutes and then loaded onto the Dako Autostainer.

Tissue samples were stained using a Dako Autostainer. For example, 3% hydrogen peroxide was added for 5 minutes to deparafinized tissue samples followed by a buffer wash. Next, Dako Proteinase K was added for 10 minutes to the tissue sample followed by a buffer wash. Diluted antibody (diluted with Dako Antibody diluent) was then added for 30 minutes followed by a buffer wash. Next, Envision+Dual Link was added for 30 minutes followed by a buffer wash. DAB+substrate-chromogen was then added for 5 minutes followed by a wash with deionized water.

Next, tissue samples were counterstained with methyl green. Stained slides were placed into a metal slide rack which was then placed into a glass dish containing methyl green counterstained for 3 minutes. The slide rack was then dipped into deionized water to remove some of the methyl green. Next, excess water was removed by blotting on a paper towel. Slides were then dehydrated by dipping the slide rack into butyl alcohol 5-10 times. This wash step was repeated through two changes of butyl alcohol. Next, slides were dipped into xylene 10 times. This wash step was repeated through two changes of xylene. Slides were then coverslipped.

Next, image analysis was preformed using the AccuMed AcCell Image Analyzer. A control slide of known value was run prior to the specimens. Quantitation of the control slide was within ±5% of the known value (the control slide must be within this parameter to continue with the analysis). The control's OD value was recorded on the AcCell control sheet. Barcode labels were prepared and applied to all slides including a negative control slide. The TMD accession number was written on the side of the label. The first slide was placed under the microscope objective and “cytoplasmic or membranous markers” was selected from the “Stain Type” drop-down menu. “Tissue” was selected from the “Slide Type” drop-down menu (“Tissue Control” was selected for the negative control slide). Fields representative of stained tumor cells were marked by centering the ocular crosshair on the desired area of the slide and clicking “Mark Points”. The above step was repeated until 10 fields to be quantified were marked for each slide. Red dots appeared in the slide status window to indicate the marked points on the slide. After 10 points were marked for quantitation, the slide was returned to the slide load position. For each antibody, a negative control slide stained with that particular antibody was used. The negative control slide was positioned so that a part of the specimen was visible in the “Live Display” window. The slide was adjusted so that a blank area was visible in the “Live Display” window. The light intensity was adjusted to within the proper range by clicking on the “Adjust Intensity” box (the software displays when the light is within the proper range). At this point, the “Start” button was clicked to begin scanning. From this point, the process was completely automated. The “Quit” button was clicked once all the slides were analyzed to exit the program.

### Example 2

### Detection and Quantitation of p95 in Breast Tumor Samples

Twenty breast tumor samples with known ErbB2 expression that ranged from negative to high expression were used to determine the amount (e.g., expression level) of p95 in the sample. These samples indicated in Table 1 below are presented with an original diagnosis which consisted of either a visual score or a FISH score.

For each of the twenty samples, IHC was preformed using an antibody to the Her2 ICD (A0485) and an antibody to the Her2 ECD (TAB250-CBL772). Optimal dilutions of each antibody were chosen based on IHC staining of cell pellets and a 3+ ErbB2 breast tumor. Values for expression of the Her2 ICD and Her2 ECD are expressed in OD units. The difference (ODₚ₉₅) was calculated by the formula: OD_{ICD}-OD_{ECD}=ODₚ₉₅, Visual scores and quantitative OD values for A0485 and TAB250-CBL772 are indicated in Table 1. Additionally, the percentage of HER2 ICD detected by A0485 in the tumor samples that is present as in its cleaved p95 form was determined by dividing the ODₚ₉₅ by the OD_{ICD}. In four cases, patient numbers 1, 4, 9 and 12, the original score was low (0 or 1+), however these samples were scored as high (2+ or 3+) by A0485 (patient numbers 1, 4, 9 and 12) and TAB250-CBL772 (Pt. #9). Conversely, in 2 samples the original score was high (3+), however, these 2 samples were scored as low by both A0485 and TAB250-CBL772 (patient numbers 7 and 8). Without wishing to be bound by a theory of the invention, it is believed that the difference in results may be explained by the use of different antibodies in the original IHC analysis.

TABLE 1
p95 expression in 20 breast tumor samples

| | | | | | | | | % of HER2 ICD |
|---|---|---|---|---|---|---|---|---|
| | | | | | | | | detected by A0485 |
| | | Original | | | Tab250- | TAB250- | | that is present as |
| | Original | ErbB2 | A0485 | A0485 | CBL772 | CBL772 | Difference | p95 cleavage |
| Pt. # | score | FISH | (OD_{ICD}) | (score) | (OD_{ECD}) | (score) | (ODₚ₉₅) | product |
| 1 | 1+ | | 29 | 3+ | 7 | 1+ | 22 | 75.9% |
| 2 | | Amplified | 57 | 3+ | 46 | 3+ | 11 | 19.3% |
| 3 | | Non- | 19 | 1+ | 6 | 1+ | 13 | 68.4% |
| | | amplified | | | | | | |
| 4 | 0 | Non- | 34 | 3+ | 7 | 1+ | 27 | 79.4% |
| | | amplified | | | | | | |
| 5 | | Non- | 67 | 3+ | 64 | 3+ | 3 | 4.5% |
| | | amplified | | | | | | |
| 6 | | Amplified | 63 | 3+ | 40 | 3+ | 23 | 36.5% |
| 7 | 3+ | | 9 | 1+ | 9 | 1+ | 0 | 0% |
| 8 | 3+ | | 10 | 1+ | 2 | 0 | 8 | 80.0% |
| 9 | 0 | | 38 | 3+ | 34 | 3+ | 4 | 10.5% |
| 10 | 3+ | | 21 | 2+ | 10 | 2+ | 11 | 52.4% |
| 11 | 0 | | 9 | 1+ | 0 | 0 | 9 | 100.0% |
| 12 | 0 | | 15 | 2+ | 7 | 1+ | 8 | 53.3% |
| 13 | 3+ | | 57 | 3+ | 52 | 3+ | 5 | 8.8% |
| 14 | 3+ | | 16 | 3+ | 10 | 1+ | 6 | 37.5% |
| 15 | 2+ | | 6 | 2+ | 2 | 0 | 4 | 66.7% |
| 16 | | Amplified | 38 | 3+ | 33 | 3+ | 5 | 13.2% |
| 17 | 3+ | | 46 | 3+ | 38 | 3+ | 8 | 17.4% |
| 18 | | Amplified | 55 | 3+ | 49 | 3+ | 6 | 10.9% |
| 19 | | Amplified | 60 | 3+ | 46 | 3+ | 14 | 23.3% |
| 20 | | Amplified | 15 | 2+ | 11 | 2+ | 4 | 26.7% |

Next, an inter-day precision analysis was conducted on five randomly selected samples (from Table 1) to determine the reproducibility and reliability of the assay. Briefly, IHC was performed on five ErbB2 expressing tumor samples in five consecutive experiments using A0485 and TAB250-CBL772. The difference (ODₚ₉₅) was calculated by the formula: OD_{ICD}-OD_{ECD}=ODₚ₉₅ Average and standard deviation for ICD staining, ECD staining and differential is shown for each patient (Table 2). The precision data indicated that the intracellular and extracellular staining, as well as the subtractive methodology, was highly reproducible over a five day period.

TABLE 2
Inter-day Precision Data

| | | TAB250-CBL772 | Difference |
|---|---|---|---|
| Pt. #-Experiment # | A0485 (OD_{ICD}) | (OD_{ECD}) | (ODₚ₉₅) |
| 1-1 | 30 | 12 | 18 |
| 1-2 | 34 | 12 | 22 |
| 1-3 | 32 | 8 | 24 |
| 1-4 | 21 | 10 | 11 |
| 1-5 | 35 | 8 | 27 |
| Average | 30.4 | 10 | 20.4 |
| Standard Deviation | 5.5 | 2 | 6.2 |
| 6-1 | 66 | 46 | 20 |
| 6-2 | 65 | 52 | 13 |
| 6-3 | 69 | 44 | 25 |
| 6-4 | 65 | 46 | 19 |
| 6-5 | 65 | 39 | 26 |
| Average | 66 | 45.4 | 20.6 |
| Standard Deviation | 1.7 | 4.7 | 5.2 |
| 8-1 | 18 | 2 | 16 |
| 8-2 | 15 | 4 | 11 |
| 8-3 | 16 | 2 | 14 |
| 8-4 | 19 | 2 | 17 |
| 8-5 | 19 | 1 | 18 |
| Average | 17.4 | 2.2 | 15.2 |
| Standard Deviation | 1.8 | 1.1 | 2.8 |
| 13-1 | 54 | 43 | 11 |
| 13-2 | 55 | 48 | 7 |
| 13-3 | 43 | 44 | −1 |
| 13-4 | 46 | 42 | 4 |
| 13-5 | 45 | 46 | −1 |
| Average | 48.6 | 44.6 | 4 |
| Standard Deviation | 5.5 | 2.4 | 5.2 |
| 15-1 | 17 | 5 | 12 |
| 15-2 | 18 | 10 | 8 |
| 15-3 | 15 | 1 | 14 |
| 15-4 | 20 | 5 | 15 |
| 15-5 | 19 | 1 | 18 |
| Average | 17.8 | 4.4 | 13.4 |
| Standard Deviation | 1.9 | 3.7 | 3.7 |

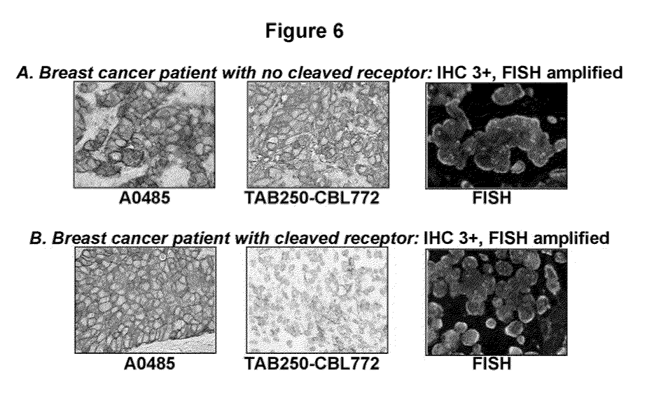

P95 analysis was additionally conducted on ten breast tumor samples with known ErbB2 FISH status (Table 3). Briefly, IHC was performed on the tumor samples using the A0485 and TAB250-CBL772 antibodies. Visual score and OD values are shown. The difference (Oₚ₉₅) was calculated by the formula: OD_{ICD}-OD_{ECD}=ODₚ₉₅. Three of the samples were non-amplified for ErbB2 and were either 0 or 1+ by TAB250-CBL772 and 2+ by A0485. ErbB2 amplification did not seem to correlate with a large p95 component by imaging analysis. Two samples which were highly amplified by FISH showed different p95 expression by imaging analysis (FIG. 6). The results of this analysis suggest that ErbB2 amplification may not necessarily indicate excessive ErbB2 cleavage.

TABLE 3
p95 expression in 10 breast tumor samples

| | | | | TAB250- | TAB250- | |
|---|---|---|---|---|---|---|
| Case | FISH | A0485 | A0485 | CBL772 | CBL772 | Difference |
| ID | ErbB2 | (OD_{ICD}) | (score) | (OD_{ECD}) | (score) | (ODₚ₉₅) |
| 1 | Amplified | 59 | 3+ | 51 | 3+ | 8 |
| 2 | Non- | 13 | 2+ | 3 | 1+ | 10 |
| | amplified | | | | | |
| 3 | Amplified | 52 | 3+ | 48 | 3+ | 4 |
| 4 | Non- | 26 | 2+ | 3 | 0 | 23 |
| | amplified | | | | | |
| 5 | Amplified | 30 | 3+ | 6 | 1+ | 24 |
| 6 | Amplified | 55 | 3+ | 44 | 3+ | 11 |
| 7 | Amplified | 42 | 3+ | 43 | 3+ | −1 |
| 8 | Non- | 17 | 2+ | 5 | 1+ | 12 |
| | amplified | | | | | |
| 9 | Amplified | 37 | 3+ | 8 | 2+ | 29 |
| 10 | Amplified | 53 | 3+ | 8 | 2+ | 45 |

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

What is claimed is:

1. A method for determining an amount of p95 in a biological sample, said method comprising:
(a) assaying the biological sample for expression of HER2 intracellular domain (ICD);
(b) assaying the biological sample for expression of HER2 extracellular domain (ECD);
(c) quantitating the expressed amount of the HER2 ICD and the expressed amount of the HER2 ECD in the biological sample; and
(d) determining the amount of p95 expressed in the biological sample based on a difference between the expressed amount of the HER2 ICD and the expressed amount of the HER2 ECD.

2. The method of claim 1, wherein biological sample is a tumor biopsy.

3. The method of claim 1, wherein the biological sample is an aspirate.

4. The method of claim 1, wherein the HER2 ICD and the HER2 ECD are assayed using a detectably labeled antibody specific for the HER2 ICD and a detectably labeled antibody specific for the HER2 ECD.

5. The method of claim 4, wherein the antibody is a monoclonal antibody.

6. The method of claim 4, wherein the label is a chromagen or fluorophore.

7. The method of claim 1, wherein the step of assaying is performed by immunohistochemistry (IHC) or western blot.

8. The method of claim 7, wherein the step of quantitating the expression of HER2 ICD and the expression of HER2 ECD in the normal cells and the tumor cells is preformed by image analysis.

9. The method of claim 8, wherein the step of quantitating the expression of the HER2 ICD and the expression of the HER2 ECD is preformed in a defined cellular area.

10. The method of claim 9, wherein the defined cellular area is the nucleus.

11. The method of claim 9 wherein the defined cellular area is the cytoplasm.

12. The method of claim 9, wherein the defined cellular area is the membrane.

13. The method of claim 9, wherein the amount of the expression of the HER2 ICD and the amount of expression of the HER2 ECD is quantitated from an average optical density (OD) of expression of the HER2 ICD and the HER2 ECD per pixel in the defined cellular area.

14. The method of claim 13, wherein the amount of the expression of the HER2 ICD and the amount of expression of the HER2 ECD is quantitated from an average OD determined on a per cell basis in the defined cellular area.

15. The method of claim 14, wherein the average OD on a per cell basis is obtained by dividing the average OD for the defined cellular area by a number of nuclei in the defined cellular area.

16. The method of claim 13, wherein the amount of p95 in the biological sample is determined by subtracting the average OD obtained from the HER2 ICD from the average OD obtained from the HER2 ECD.

17. The method of claim 1, wherein the HER2 ICD comprises SEQ ID NO: 3.

18. The method of claim 1, wherein the HER2 ECD comprises SEQ ID NO: 2.

19. A method for determining an amount of p95 in a biological sample, said method comprising:
a. assaying the biological sample for expression of HER2 ICD with an antibody specific for the HER2 ICD;
b. assaying the biological sample for expression of HER2 ECD with an antibody specific for the HER2 ECD;
c. quantitating the expressed amount of the HER2 ICD and the expressed amount of the HER2 ECD in the biological sample from an average optical density (OD) of expression of the HER2 ICD and the HER2 ECD on a per cell basis in the defined cellular area, wherein the average OD on a per cell basis is obtained by dividing the average OD for the defined cellular area by a number of nuclei in the defined cellular area; and
d. determining the amount of p95 expressed in the biological sample based on a difference between the expressed amount of the HER2 ICD and the expressed amount of the HER2 ECD, wherein the amount of p95 in the biological sample is determined by subtracting the average OD obtained from the HER2 ICD from the average OD obtained from the HER2 ECD.

20. A method for determining an amount of HER2 ICD in a cell that is present as a p95 cleavage product and/or a p95 truncated translation product, the method comprising:
a. assaying the biological sample for expression of HER2 ICD;
b. assaying the biological sample for expression of HER2 ECD;
c. quantitating the expressed amount of the HER2 ICD and the expressed amount of the HER2 ECD in the biological sample; and
d. determining the amount of p95 expressed in the biological sample based on a difference between the expressed amount of the HER2 ICD and the expressed amount of the HER2 ECD; and
e. determining the amount of HER2 ICD that is present as a p95 cleavage product and/or a p95 truncated translation product by comparing the amount of HER2 ICD detected in the biological sample to the amount of p95 detected in the biological sample.

21. The method of claim 19, wherein the amount of HER2 ICD that is present as the p95 cleavage product and/or the p95 truncated translation product is calculated by dividing the amount of HER2 ICD by the amount of p95 detected in the biological sample.

22. A method for predicting responsiveness of a subject with a disease or disorder to a receptor tyrosine kinase inhibitor, said method comprising:
a. obtaining a biological sample from the subject;
b. assaying the biological sample for expression of HER2 ICD;
c. assaying the biological sample for expression of HER2 ECD;
d. quantitating an expressed amount of the HER2 ICD and the expressed amount of the HER2 ECD in the biological sample;
e. calculating the amount of p95 in the biological sample based on a difference between the expressed amount of the HER2 ICD and the HER2 ECD;
f. comparing the amount of p95 expressed in the biological sample to an amount of p95 expressed in a control sample or comparing the amount of p95 expressed in the biological sample to a threshold; and
g. determining that the subject is responsive to a receptor tyrosine kinase inhibitor where the amount of p95 expressed in the biological sample is less than the amount of p95 expressed in the control sample or less than the threshold or determining that the subject is not responsive to a receptor tyrosine kinase inhibitor where the amount of p95 expressed in the biological sample is greater than the amount of p95 expressed in the control sample or greater than the threshold.

23. The method of claim 22, wherein biological sample is a tumor biopsy.

24. The method of claim 22, wherein the biological sample is an aspirate.

25. The method of claim 22, wherein the HER2 ICD and the HER2 ECD are assayed using a detectably labeled antibody specific for the HER2 ICD and a detectably labeled antibody specific for the HER2 ECD.

26. The method of claim 25, wherein the antibody is a monoclonal antibody.

27. The method of claim 25, wherein the label is a chromagen or fluorophore.

28. The method of claim 22, wherein the step of assaying is performed by immunohistochemistry (IHC) or western blot.

29. The method of claim 22, wherein the step of quantitating the expression of HER2 ICD and the expression of HER2 ECD in the normal cells and the tumor cells is preformed by image analysis.

30. The method of claim 29, wherein the step of quantitating the expression of the HER2 ICD and the expression of the HER2 ECD is preformed in a defined cellular area.

31. The method of claim 30, wherein the defined cellular area is the nucleus.

32. The method of claim 30, wherein the defined cellular area is the cytoplasm.

33. The method of claim 30, wherein the defined cellular area is the membrane.

34. The method of claim 30, wherein the amount of the expression of the HER2 ICD and the amount of expression of the HER2 ECD is quantitated from an average optical density (OD) of expression of the HER2 ICD and the HER2 ECD per pixel in the defined cellular area.

35. The method of claim 34, wherein the amount of the expression of the HER2 ICD and the amount of expression of the HER2 ECD is quantitated from an average OD determined on a per cell basis in the defined cellular area.

36. The method of claim 35, wherein the average OD on a per cell basis is obtained by dividing the average OD for the defined cellular area by a number of nuclei in the defined cellular area.

37. The method of claim 34, wherein the amount of p95 in the biological sample is determined by subtracting the average OD obtained from the HER2 ICD from the average OD obtained from the HER2 ECD.

38. The method of claim 22, wherein the HER2 ICD comprises SEQ ID NO: 3.

39. The method of claim 22, wherein the HER2 ECD comprises SEQ ID NO: 2.

40. The method of claim 22, wherein the receptor tyrosine kinase inhibitor is an antibody.

41. The method of claim 40, wherein the antibody is a monoclonal antibody.

42. The method of claim 41, wherein the monoclonal antibody is cetuximab (Erbitux), panitumumab, zalutumumab, nimotuzumab or matuzumab.

43. The method of claim 22, wherein the receptor tyrosine kinase inhibitor is a small molecule inhibitor.

44. The method of claim 43, wherein the small molecule inhibitor is gefitinib, erlotinib or lapatinib.

45. The method of claim 22, wherein the disease or disorder is cancer.

46. The method of claim 45, wherein the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

47. The method of claim 22, wherein the subject is a cancer patient.

48. The method of claim 22, wherein the control sample is obtained from the patient from which the biological sample was obtained.

49. The method of claim 22, wherein the control sample is obtained from a different patient from which the biological sample was obtained.

50. The method of claim 22, wherein the control sample is non-cancerous cells or tissue.

51. The method of claim 22, wherein the threshold is set as a maximum amount of p95 in a biological sample in which a subject is responsive to treatment with a receptor tyrosine kinase inhibitor.

52. A method for treating a subject with a disease or disorder with a receptor tyrosine kinase inhibitor, the method comprising:
a. obtaining a biological sample from the subject;
b. assaying the biological sample for expression of HER2 ICD;
c. assaying the biological sample for expression of HER2 ECD;
d. quantitating an expressed amount of the HER2 ICD and an expressed amount of the HER2 ECD in the biological sample;
e. calculating the amount of p95 expressed in the biological sample based on a difference between the expressed amount of the HER2 ICD and the expressed amount of the HER2 ECD;
f. comparing the amount of p95 expressed in the biological sample to an amount of p95 expressed in a control sample or comparing the amount of p95 expressed in the biological sample to a threshold;
g. determining that the subject is responsive to a receptor tyrosine kinase inhibitor where the amount of p95 expressed in the biological sample is less than the amount of p95 expressed in the control sample or less than the threshold; and
h. administering the receptor tyrosine kinase inhibitor to the subject.

53. The method of claim 52, wherein biological sample is a tumor biopsy.

54. The method of claim 52, wherein the biological sample is an aspirate.

55. The method of claim 52, wherein the HER2 ICD and the HER2 ECD are assayed using a detectably labeled antibody specific for the HER2 ICD and a detectably labeled antibody specific for the HER2 ECD.

56. The method of claim 55, wherein the antibody is a monoclonal antibody.

57. The method of claim 55, wherein the label is a chromagen or fluorophore.

58. The method of claim 52, wherein the step of assaying is performed by immunohistochemistry (IHC) or western blot.

59. The method of claim 52, wherein the step of quantitating the expression of HER2 ICD and the expression of HER2 ECD in the normal cells and the tumor cells is preformed by image analysis.

60. The method of claim 59, wherein the step of quantitating the expression of the HER2 ICD and the expression of the HER2 ECD is preformed in a defined cellular area.

61. The method of claim 60, wherein the defined cellular area is the nucleus.

62. The method of claim 60, wherein the defined cellular area is the cytoplasm.

63. The method of claim 60, wherein the defined cellular area is the membrane.

64. The method of claim 60, wherein the amount of the expression of the HER2 ICD and the amount of expression of the HER2 ECD is quantitated from an average optical density (OD) of expression of the HER2 ICD and the HER2 ECD per pixel in the defined cellular area.

65. The method of claim 64, wherein the amount of the expression of the HER2 ICD and the amount of expression of the HER2 ECD is quantitated from an average OD determined on a per cell basis in the defined cellular area.

66. The method of claim 65, wherein the average OD on a per cell basis is obtained by dividing the average OD for the defined cellular area by a number of nuclei in the defined cellular area.

67. The method of claim 64, wherein the amount of p95 in the biological sample is determined by subtracting the average OD obtained from the HER2 ICD from the average OD obtained from the HER2 ECD.

68. The method of claim 52, wherein the HER2 ICD comprises SEQ ID NO: 3.

69. The method of claim 52, wherein the HER2 ECD comprises SEQ ID NO: 2.

70. The method of claim 52, wherein the receptor tyrosine kinase inhibitor is an antibody.

71. The method of claim 70, wherein the antibody is a monoclonal antibody.

72. The method of claim 71, wherein the monoclonal antibody is cetuximab (Erbitux), panitumumab, zalutumumab, nimotuzumab or matuzumab.

73. The method of claim 52, wherein the receptor tyrosine kinase inhibitor is a small molecule inhibitor.

74. The method of claim 73, wherein the small molecule inhibitor is gefitinib, erlotinib or lapatinib.

75. The method of claim 52, wherein the disease or disorder is cancer.

76. The method of claim 75, wherein the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

77. The method of claim 52, wherein the subject is a cancer patient.

78. The method of claim 52, wherein the threshold is set as a maximum amount of p95 in a biological sample in which a subject is responsive to treatment with a receptor tyrosine kinase inhibitor.

79. A method for selecting subjects for inclusion/exclusion in a clinical trial, said method comprising:
a. obtaining a biological sample from the subjects;
b. assaying the biological samples for expression of HER2 ICD;
c. assaying the biological samples for expression of HER2 ECD;
d. quantitating the expressed amount of HER2 ICD and the expressed amount of HER2 ECD in the biological samples;
e. determining the amount of p95 in the biological samples based on a difference between the expressed amount of the HER2 ICD and the HER2 ECD;
f. selecting subjects for inclusion/exclusion in the clinical trial where the amount of p95 detected in the biological sample from each subject is less than/greater than a control amount of p95.

80. The method of claim 79, wherein subjects are excluded from the clinical trial where the amount of p95 detected in their biological sample is greater than the control amount of p95.

81. The method of claim 79, wherein subjects are included in the clinical trial where the amount of p95 detected in their biological sample is equal to or less than a control amount of p95.

82. The method of claim 79, wherein the control amount of p95 is that amount of p95 detected in subjects that are tumor free.

83. The method of claim 79, wherein the clinical trial is a phase I, phase II, phase III or phase IV clinical trial.


SEQUENCE LISTING (3 sequences)

SEQ ID NO: 1 - protein, 1255 aa, human [sequence omitted]

SEQ ID NO: 2 - protein, 675 aa, human
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILI

SEQ ID NO: 3 - protein, 580 aa, human
KRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV